(19) United States
(12) Patent Application Publication
(10) Pub. No.: US 2013/0123891 A1
(43) Pub. Date: May 16, 2013

(54) HIGH DENSITY TERMINAL CONTACTS FOR STIMULATION LEAD AND STIMULATION SYSTEM EMPLOYING THE SAME, AND METHOD OF STIMULATION LEAD FABRICATION

(71) Applicant: John Swanson, Portland, OR (US)

(72) Inventor: John Swanson, Portland, OR (US)

(21) Appl. No.: 13/607,280

(22) Filed: Sep. 7, 2012

Related U.S. Application Data
(63) Continuation of application No. 12/361,807, filed on Jan. 29, 2009, now abandoned.
(60) Provisional application No. 61/025,369, filed on Feb. 1, 2008.

(51) Int. Cl. A61N 1/04; H05K 3/12; H05K 3/02; H05K 3/00; H05K 3/06

(52) U.S. Cl. CPC A61N 1/04; H05K 3/00; H05K 3/06; H05K 3/027; H05K 3/12
USPC 607/115; 29/829; 29/847; 29/846

(57) ABSTRACT

In one embodiment, a method of fabricating a lead comprises: providing a lead body comprising a plurality of conductive wires; providing a flex film connector structure, the flex film connector structure comprising a plurality of conductive pads on a first portion of the flex film connector structure, a plurality of contacts on a second portion of the flex film connectors, and a plurality of traces electrically connecting the plurality of conductive pads with the plurality of contacts; placing the first portion of the flex film connector adjacent to a cross-section of one end of the lead body; electrically coupling the plurality of conductive pads of the flex film connector structure to the plurality of conductive wires at the one end of the lead body; and wrapping the second portion of the flex film connector structure about the lead body to form a plurality of electrical contacts.

### CROSS-REFERENCE TO RELATED APPLICATIONS

This application is a continuation of U.S. application Ser. No. 12/361,807, filed Jan. 29, 2009, pending, which claims the benefit of U.S. Provisional Application No. 61/025,369, filed Feb. 1, 2008, the disclosures of which are fully incorporated herein by reference for all purposes.

### TECHNICAL FIELD

The present application is generally related to a design for electrode and/or terminal contacts of a stimulation lead, a stimulation system, and a method of fabrication of a stimulation lead.

### BACKGROUND

Neurostimulation systems are devices that generate electrical pulses and deliver the pulses to nerve tissue to treat a variety of disorders. Spinal cord stimulation (SCS) is an example of neurostimulation in which electrical pulses are delivered to nerve tissue in the spine, typically for the purpose of treating chronic pain. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

Neurostimulation systems generally include a pulse generator and one or several leads. The pulse generator is typically implemented using a metallic housing that encloses circuitry for generating the electrical pulses. The pulse generator is usually implanted within a subcutaneous pocket created under the skin by a physician. The leads are used to conduct the electrical pulses from the implant site of the pulse generator to the targeted nerve tissue. The leads typically include a lead body of an insulative polymer material with embedded wire conductors extending through the lead body. Electrodes on a distal end of the lead body are coupled to the conductors to deliver the electrical pulses to the nerve tissue.

Leads are typically adapted to couple with electrical connectors enclosed within a header structure of the implantable pulse generator. “Terminal” contacts are fabricated on a proximal end of the lead to facilitate the electrical coupling. The terminal contacts are usually fabricated in substantially the same manner as the fabrication of electrodes on a distal end of the lead. Typically, an aperture is made in the lead body to expose a conductive wire within the lead body, a conductive connector is disposed within the aperture, and a metal ring is swaged or crimped around the lead body and is placed in electrical contact with the conductive connector.

### SUMMARY

In one embodiment, a method of fabricating a lead comprises: providing a lead body comprising a plurality of conductive wires; providing a flex film connector structure, the flex film connector structure comprising a plurality of conductive pads on a first portion of the flex film connector structure, a plurality of contacts on a second portion of the flex film connectors, and a plurality of traces electrically connecting the plurality of conductive pads with the plurality of contacts; placing the first portion of the flex film connector adjacent to a cross-section of one end of the lead body; electrically coupling the plurality of conductive pads of the flex film connector structure to the plurality of conductive wires at the one end of the lead body; and wrapping the second portion of the flex film connector structure about the lead body to form a plurality of electrical contacts.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 depicts a flex film connector according to one representative embodiment.

FIG. 2 depicts one end of a stimulation lead according to one representative embodiment.

FIGS. 3A and 3B depict respective flowcharts for fabricating a flex film connector according to some representative embodiments.

FIG. 4 depicts a flowchart for fabrication of a stimulation lead according to one representative embodiment.

FIG. 5 depicts a stimulation system according to one representative embodiment.

FIG. 6 depicts an annular portion of a flex film connector according to one representative embodiment.

FIG. 7 depicts a rectangular portion of a flex film connector according to one representative embodiment.

### DETAILED DESCRIPTION

Some representative embodiments are directed to a stimulation lead that comprises a flex film with conductive bands to couple with the electrical connectors of an implantable pulse generator. By employing electrical contacts formed on a flex film substrate, the spacing between electrical contacts can be relatively close. Accordingly, a smaller header design can be employed and, hence, a smaller implantable pulse generator can be achieved. In other embodiments, the stimulation electrodes adapted to provide electrical pulses to tissue of a patient are formed on a flex film substrate. The use of a flex film design for terminals and/or electrodes may allow the lead fabrication process to occur in a more efficient manner.

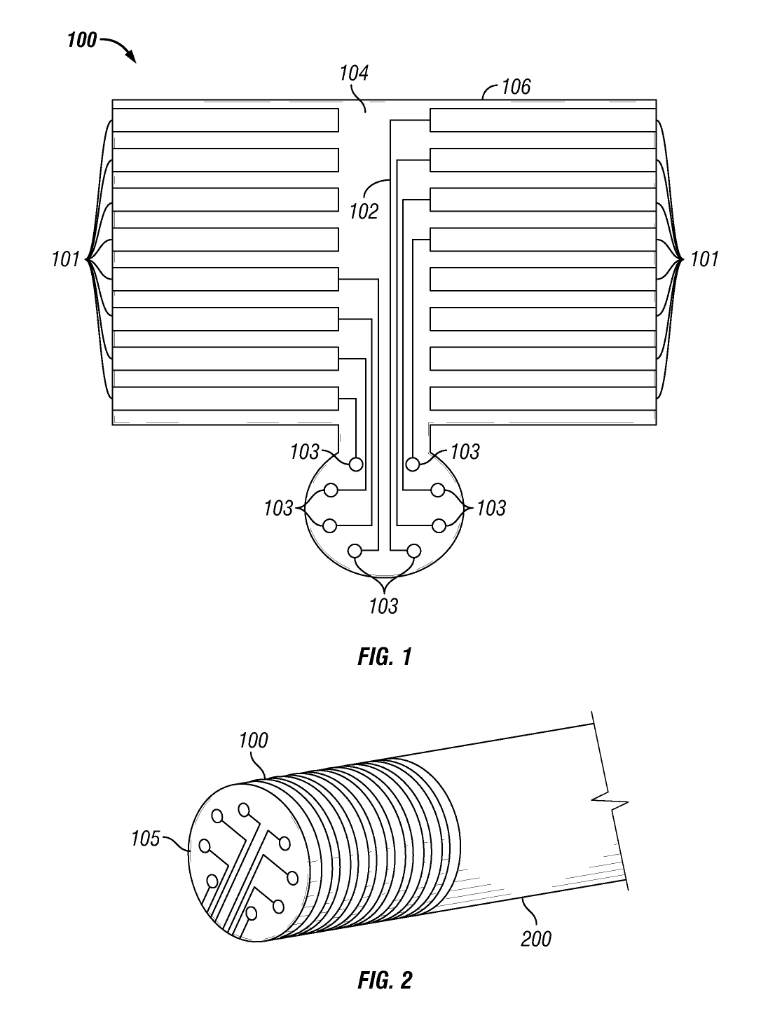

FIG. 1 depicts flex film connector 100 that comprises a plurality of electrical contacts 101 according to one representative embodiment. Flex film connector 100 is preferably implemented by providing metal contacts 101, traces 102, and pads 103 adjacent to insulative substrate 104. Insulative substrate 104 preferably comprises annular portion 105 and rectangular portion 106. The insulative backing may be formed of urethane material, polyimide material, or preferably polyetheretherketone (PEEK) material, liquid crystal polymer (LCP) material, polyetherketoneketone (PEKK) material, as examples. The various metal components can be disposed on the insulative backing using any suitable number of techniques. For example, a continuous metal film may be bonded to the insulative backing and the various metal components can be defined by etching away metal between the discrete components using a programmable YAG laser system. Photolithographic and metallization fabrication processes can also be utilized to define and deposit the various metal components onto insulative substrate 104. In another alternative embodiment, micro-printing is employed to deposit the conductive material.

As shown in FIG. 1, flex film connector 100 comprises annular structure 105 on which a plurality of pads 103 are formed, deposited, bonded, or otherwise provided. Annular structure 105 is approximately sized according to the cross-section of a stimulation lead body to which flex film connector 100 will be attached. Pads 103 on annular structure 105 are preferably arranged according to the same pattern as the conductive wires within the stimulation lead. In some embodiments, the size of pads 103 is relatively large as compared to the size of the wire conductors to provide an amount of tolerance for variability of the position of the conductive wires within a lead. In one embodiment, a hole or slit (not shown) may be provided in each pad 103. A respective wire of the lead body may be exposed and threaded through the hole or slit in each pad 103. The respective wire can then be welded or soldered to pad 103 to facilitate the electrical connection.

Each pad 103 is, in turn, electrically coupled to a respective metal contact 101 by a corresponding trace 102 (only one trace 102 is annotated in FIG. 1 for the sake of clarity). In the embodiment shown in FIG. 1, each contact 101 is separated into two portions on the left and right sides of rectangular portion 106 of insulative substrate 104. One side of each contact 101 is intended to be electrically coupled to the other side when flex film connector 100 is attached to the stimulation lead.

FIG. 2 depicts flex film connector 100 coupled to stimulation lead 200. Annular structure 105 is disposed at the proximal end of the stimulation lead. Pads 103 are preferably electrically coupled to the conductive wires (not shown) within stimulation lead 200. The remaining portion of flex film connector 100 is wrapped around the body of stimulation lead 200 such that contacts 101 substantially circumscribe the lead body. Although flex film connector 100 as shown in FIG. 1 is only adapted to provide eight terminal contacts, any suitable number (e.g., sixteen or thirty-two) of terminal contacts may be employed according to alternative embodiments.

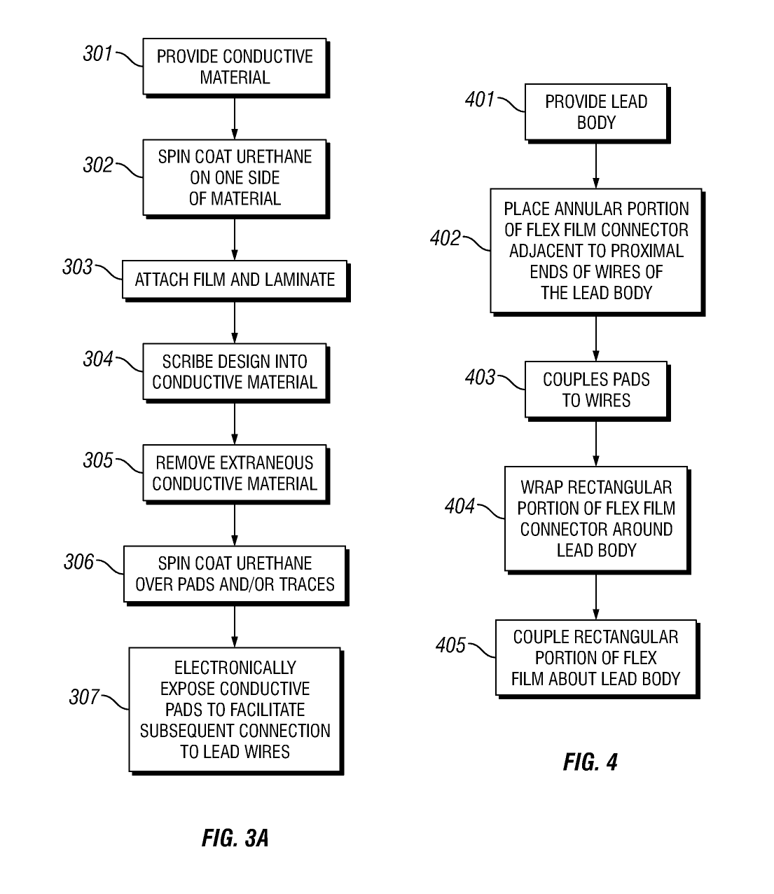

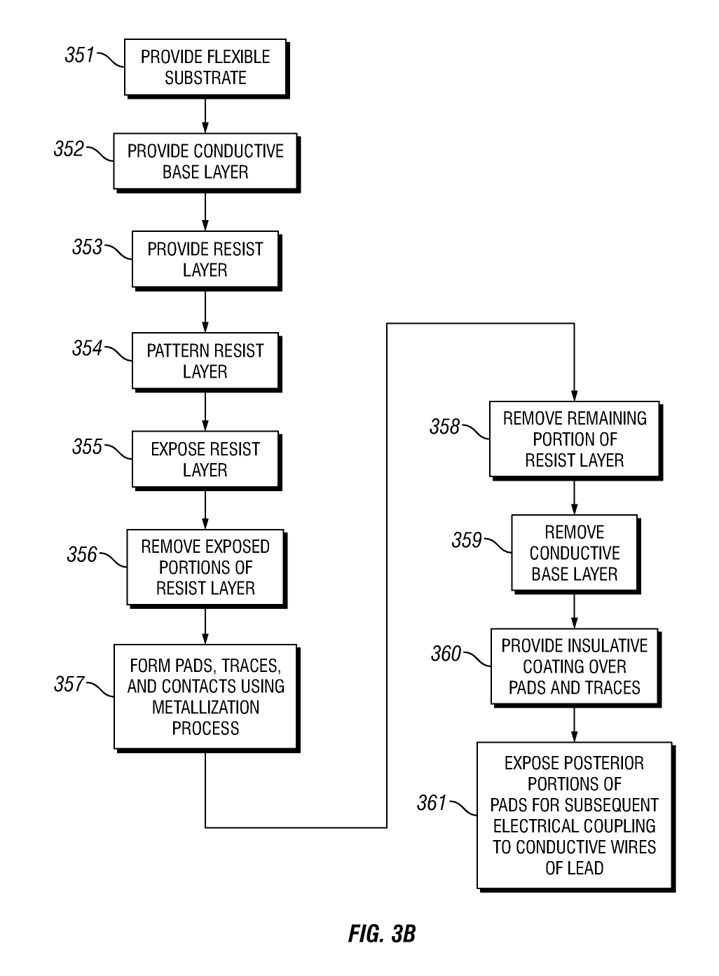

FIG. 3A depicts a flowchart for fabricating flex film connector 100 according to one representative embodiment. In 301, a rectangle or other suitable portion of conductive material is provided. Although the following discussion only refers to fabrication of a single flex film connector 100, multiple flex film connectors 100 can be fabricated in parallel on suitably sized portion of conductive material according to some representative embodiments. The conductive material can be gold, conductive composite materials, medical grade stainless steel, platinum iridium, and/or the like. The thickness of the conductive material is selected to allow the conductive material to be relatively flexible. As an example, a suitable thickness for stainless steel would be about 25.4 microns (1 mil).

In 302, a thin coating of urethane (or a similar polymer) is spin coated on one side of the conductive material for the purpose of achieving a surface with greater adhesive qualities. In 303, a urethane film (or any other suitable polymer) is applied to the same side as the spin coat and is laminated to the conductive material.

In 304, contacts 101, traces 102, and pads 103 are created by scribing their respective forms in the conductive material using a suitable laser (e.g., a programmable YAG laser system). Specifically, the programmable YAG laser system traces its output beam along the contour defined between the various metal components. The application of the laser energy separates the desired metal components from the extraneous material. In 305, the extraneous metal material is optionally removed or peeled away from the insulative backing, leaving contacts 101, traces 102, and pads 103 in place.

In 306, a thin top coat of insulative material is applied to portions of the assembly. For example, a spin coat of an insulative material can be applied over annular structure 105 and/or traces 102. Contacts 101 are left exposed, since contacts 101 are intended to make electrical contact within the header of an implantable pulse generator.

In 307, electrical connections are made for the purpose of exposing portions of pads 103 on the posterior side of flex film connector 100 to permit electrical coupling with the lead wires of a stimulation lead (see the description of the flowchart of FIG. 4 below). Selected portions of the insulative material could be removed to expose portions of pads 103. A CO₂ laser, a YAG laser, other suitable laser could be utilized for this purpose. Alternatively, conductive material could be fabricated on the backside of flex film connector and typical via processing can be used to facilitate the subsequent electrical coupling with the conductors of the lead.

FIG. 3B depicts a flowchart for fabricating flex film connector 100 according to one representative embodiment. In 351, a flexible substrate is provided. The substrate can be formed of urethane material, polyimide material, or preferably polyetheretherketone (PEEK) material, liquid crystal polymer (LCP) material, polyetherketoneketone (PEKK) material, as examples. In 352, a conductive base layer is provided to facilitate subsequent provision of the metal that will be utilized to form the traces, pads, and contacts.

A resist layer is provided (353) and the resist layer is patterned (354). The patterned resist layer is exposed to radiation at an appropriate wavelength (355). The exposed portions of the resist layers are removed (356). In the removed areas, pads, traces, and contacts are formed using a suitable metallization process (357). The remaining portion of the resist layer is removed (358) and the remaining conductive base layer is removed (359) leaving the substrate with electrically isolated sets of pads, traces, and contacts on the substrate. An insulative coating is preferably provided over the traces and pads (360). Posterior portions of the pads are exposed for subsequent electrical coupling to conductive wires of a stimulation lead (361).

FIG. 4 depicts a flowchart for fabricating a stimulation lead according to one representative embodiment. In 401, a stimulation lead body is provided. Any known or later developed stimulation lead body can be provided. An example of a lead body that can be employed according to one representative embodiment is described in U.S. Patent Application Publication No. 20050027339, entitled “System and method for providing a medical lead body,” which is incorporated herein by reference. The lead body may comprise a single layer of conductive wires or multiple layers. If the stimulation lead comprises multiple layers of conductive wires, annular portion 105 would be modified to arrange pads 103 in a pattern corresponding to the cross-sectional arrangement of conductive wires in the lead body. The conductive wires within the lead body may be disposed in a linear arrangement, a helical manner, or any other suitable pattern along the longitudinal length of the lead body.

In 402, annular portion 105 of flex film connector 100 is placed over the proximal or distal face of the stimulation lead such that pads 103 are placed immediately adjacent to the respective ends of the conductive wires within the stimulation lead. In 403, pads 103 are mechanically and electrically coupled to the conductive wires. Laser welding, resistive welding, conductive epoxy bonding, or any other suitable technique may be employed.

In 404, rectangular portion 106 of flex film connector 100 is wrapped about the lead body. In 405, the rectangular portion 106 of flex film connection 100 is suitable coupled or bonded to the lead body, e.g., glued thereto, to mechanically retain flex film connector 100 in an annular manner about the lead body. In another embodiment, respective distal portions of contacts 101 could be welded to each other about the lead body.

Electrical contacts are preferably fabricated on the other end of the stimulation lead. The same techniques can be used to create the other electrical contacts using another flex film connector 100. Alternatively, a conventional or other process may be utilized to fabricate the other electrical contacts. An example of a conventional electrode contact fabrication technique is described in U.S. Pat. No. 6,216,045, entitled “Implantable lead and method of manufacturing,” which is incorporated herein by reference. The electrical contacts on the other end of the stimulation lead could also be disposed on a paddle structure or other structure suitable for stimulation of tissue of a patient.

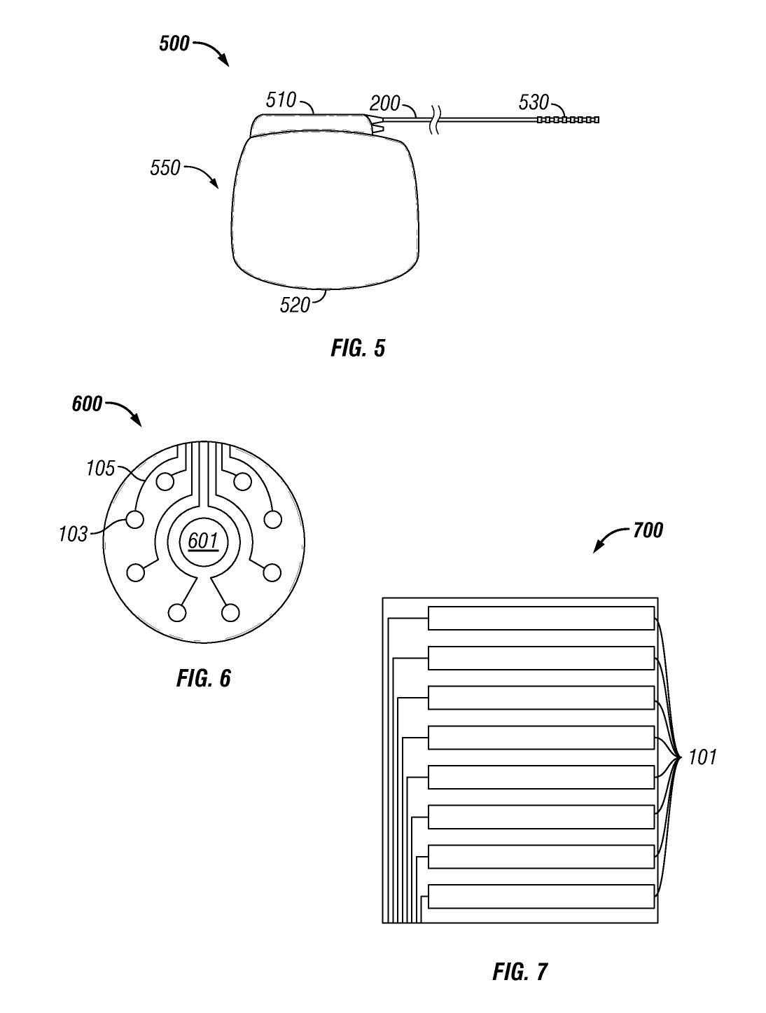

FIG. 5 depicts stimulation system 500 according to one representative embodiment. As shown in FIG. 5, lead 200 is coupled to one of apertures in header 510 of implantable pulse generator (IPG) 550. Each aperture is designed to hold electrical connectors or connections that couple to terminal contacts 101 of a respective lead 200 or an extension lead. The size of header 510 may be significantly reduced, since the density of contacts 101 of lead 200 is relatively high. Accordingly, the overall size of IPG 550 can be reduced. The electrical connectors in each aperture electrically couple to internal components contained within the sealed portion 520 of IPG 550. The sealed portion 520 contains the pulse generating circuitry, communication circuitry, control circuitry, and battery (not shown) within an enclosure to protect the components after implantation within a patient. The control circuitry controls the pulse generating circuitry to apply varying pulses to the patient via electrodes 530 of lead 200 according to multiple parameters (e.g., amplitude, pulse width, frequency, etc.). As previously mentioned, electrodes 530 could be also fabricated using a flex film connector 100 according to some representative embodiments. The parameters are set by an external programming device (not shown) via wireless communication with IPG 550.

FIG. 6 depicts another annular portion 600 suitable for a flex film connector 100 according to another representative embodiment. As shown in FIG. 6, annular portion 600 comprises aperture 601 in the insulative substrate. In one embodiment, when annular portion 600 is electrically coupled to the conductive wires of a stimulation lead, a cap structure could be pressed against annular portion 600 and a portion of the cap structure inserted through the aperture 601 and into the stylet lumen of the stimulation lead. Suitable adhesive could be employed to facilitate mechanical coupling. In such a manner, the cap structure could be utilized to provide mechanical protection of annular portion 600.

FIG. 7 depicts rectangular portion 700 suitable for a flex film connector 100 according to another representative embodiment. As shown in FIG. 700, rectangular portion 700 is asymmetric with all of the contacts 101 on one side of portion 700. Depending upon the implementation of the internal electrical connectors of the header of the implantable pulse generator, contacts 101 need not necessarily completely circumscribe the lead body. For example, the length of contacts 101 could be selected to traverse one-half of the circumference of the lead body (if desired).

In another embodiment, flex film connector 100 could be adapted to implement the electrode contacts of a stimulation lead. Flex film connector 100 would preferably size contacts 101 to function as electrodes in such a manner to permit operation according to appropriate current density constraints. Also, the spacing of contacts 101 would be adapted to permit effective electrode selection to permit optimization of stimulation therapies upon implantation of the stimulation lead as is known in the art. In other embodiments, contacts 101 could be adapted to provide directional electrodes, i.e., individual contacts 101 would only occupy a limited radial range along the circumference of a stimulation lead. For example, contacts 101 could follow the electrode designs or patterns shown in U.S. Pat. No. 7,047,084, which is incorporated herein by reference. In another embodiment, flex film connector 100 could be used to provide the terminals for a lead extension in lieu of a stimulation lead.

Although certain embodiments have been discussed for use in providing an SCS therapy for patients, electrode contacts and/or terminal contacts can be employed for any type of stimulation system according to other embodiments. Stimulation leads having terminal contacts according to some representative embodiments may be used for cardiac applications, peripheral nerve stimulation or peripheral nerve field stimulation, deep brain or cortical stimulation, gastric pacing, and/or the like as other examples.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

1. A method of fabricating a stimulation lead for providing electrical pulses to patient tissue or a lead extension, comprising:
providing a lead body, the lead body comprising a plurality of conductive wires embedded or enclosed within insulative material;
providing a flex film connector structure, the flex film connector structure comprising a plurality of conductive pads on a first portion of the flex film connector structure, a plurality of contacts on a second portion of the flex film connectors, and a plurality of traces electrically connecting the plurality of conductive pads with the plurality of contacts;
placing the first portion of the flex film connector adjacent to a cross-section of one end of the lead body;
electrically coupling the plurality of conductive pads of the flex film connector structure to the plurality of conductive wires at the one end of the lead body, wherein after performing the electrically coupling, the first portion of the flex film connector is disposed in contact with a cross-sectional surface of the lead body at a respective end of the lead body; and
wrapping the second portion of the flex film connector structure about the lead body to form a plurality of electrical contacts, wherein the second portion of the flex film connector generally circumscribes an outer diameter of the lead body after the wrapping is performed.

2. The method of claim 1 further comprising:
welding respective portions of each contact of the plurality of contacts to each other to electrically and mechanically couple the respective portions about the lead body.

3. The method of claim 1 wherein the plurality of contacts substantially circumscribe the lead body after the wrapping is performed.

4. The method of claim 1 wherein the electrically coupling comprising:
laser welding the plurality of conductive pads to the plurality of conductive wires.

5. The method of claim 1 wherein the flex film connector comprises a polyetheretherketone (PEEK), polyetherketoneketone (PEKK), or liquid crystal polymer (LCP) substrate for holding the plurality of conductive pads, the plurality of contacts, and the plurality of traces.

6. The method of claim 1 wherein the flex film connector comprises a thin insulative coating applied over the plurality of conductive pads.

7. The method of claim 1 wherein the flex film connector comprises a thin insulative coating applied over the plurality of traces.

8. The method of claim 1 wherein providing the flex film connector structure comprises:
fabricating the plurality of conductive pads, the plurality of contacts, and the plurality of traces utilizing a photolithographic and metallization fabrication processes.

9. The method of claim 1 wherein providing the flex film connector structure comprises:
fabricating the plurality of conductive pads, the plurality of contacts, and the plurality of traces utilizing a micro-printing process.

10. The method of claim 1 wherein providing the flex film connector structure comprises:
fabricating the plurality of conductive pads, the plurality of contacts, and the plurality of traces by ablating, along one or more contours of the plurality of contacts, the plurality of conductive pads, and the plurality of traces, into a segment of conductive material using a programmable laser system.

11. The method of claim 1 wherein the plurality of electrical contacts are terminal contacts for coupling with an implantable pulse generator.

12. The method of claim 1 wherein the plurality of electrical contacts are electrodes for stimulation of patient tissue.

13. A stimulation lead for providing electrical pulses to tissue of a patient, comprising:
a lead body comprising a plurality of conductive wires embedded or enclosed within insulative material;
a first plurality of electrical contacts disposed on a first end of the lead body, wherein the first plurality of electrical contacts are electrically coupled to the plurality of conductive wires; and
a flex film connector that comprises a plurality of conductive pads on a first portion of the flex film connector, a second plurality of electrical contacts on a second portion of the flex film connector, and a plurality of traces that electrically couple the plurality of conductive pads with the second plurality of electrical contacts, wherein (i) the first portion of the flex film connector is disposed adjacent to a cross-section of the lead body at a second end of the lead body; (ii) the plurality of conductive pads are electrically coupled to the plurality of conductive wires; and (iii) the second portion of the flex film connector is wrapped about the lead body.

14. The stimulation lead of claim 13 wherein the first plurality of electrical contacts are provided on a second flex film connector.

15. The stimulation lead of claim 13 wherein the flex film connector comprises a polyetheretherketone (PEEK), polyetherketoneketone (PEKK), or liquid crystal polymer (LCP) substrate.

16. The stimulation lead of claim 13 wherein respective portions of each electrical contact of the second plurality of electrical contacts are welded to each other to electrically and mechanically couple the respective portions of each electrical contact about the lead body.

17. The stimulation lead of claim 13 wherein the plurality of conductive pads are welded to the plurality of conductive wires.

18. A stimulation system for electrically stimulating tissue of a patient, comprising:
an implantable pulse generator for generating electrical pulses; and
a stimulation lead for providing electrical pulses to tissue of a patient, comprising:
a lead body comprising a plurality of conductive wires embedded or enclosed within insulative material;
a first plurality of electrical contacts disposed on a first end of the lead body, wherein the first plurality of electrical contacts are electrically coupled to the plurality of conductive wires; and
a flex film connector that comprises a plurality of conductive pads on a first portion of the flex film connector, a second plurality of electrical contacts on a second portion of the flex film connector, and a plurality of traces that electrically couple the plurality of conductive pads with the second plurality of electrical contacts, wherein (i) the first portion of the flex film connector is disposed adjacent to a cross-section of the lead body at a second end of the lead body; (ii) the plurality of conductive pads are electrically coupled to the plurality of conductive wires; and (iii) the second portion of the flex film connector is wrapped about the lead body.

19. The stimulation system of claim 18 wherein the first plurality of electrical contacts are provided on a second flex film connector.

20. The stimulation system of claim 18 wherein the stimulation lead further comprises a cap structure covering the first portion of the flex film connector.
